(19) United States
(12) Patent Application Publication
(10) Pub. No.: US 2021/0338396 A1
(43) Pub. Date: Nov. 4, 2021

(54) Manifold for attachment to a medical or dental chair and use thereof to remove aerosols emitted form a patient during a clinical procedure

(71) Applicants: Constant Charles Crohin, Watertown, MA (US); Michele Crohin, Marblehead, MA (US)

(72) Inventors: Constant Charles Crohin, Watertown, MA (US); Michele Crohin, Marblehead, MA (US)

(21) Appl. No.: 17/220,103

(22) Filed: Apr. 1, 2021

Related U.S. Application Data
(60) Provisional application No. 62/704,289, filed on May 2, 2020.

(51) Int. Cl. A61C 17/08; A61G 15/16; A61M 1/00

(52) U.S. Cl. CPC A61C 17/08; A61M 1/87; A61G 15/16

(57) ABSTRACT

The present disclosure provides a manifold that can be easily attached to a medical or dental chair in which a patient sits and a flexible arm and funnel connected to the manifold and configured to receive a High Volume Evacuator (HVE) tube. The flexible arm which holds the funnel is configured to be sufficiently rigid to maintain its shape and position, allowing a clinical practitioner to leave the funnel in the appropriate place to capture aerosols emitted from a patient during a procedure use the negative pressure from the HVE. Thus, also provided herein is a method of using the disclosed manifold to remove aerosols emitted during a clinical procedure.

### CROSS REFERENCES TO RELATED APPLICATIONS

The present application claims the benefit and priority of U.S. application No. 62/704,289, filed 2 May 2020, which is incorporated herein by reference.

### FIELD OF INVENTION

The present invention relates generally to the field of medical and dental apparatus. More specifically the present invention relates to a manifold configured to capture potentially infectious aerosols emitted by a patient during a medical or dental procedure.

### BACKGROUND

Many dental and medical procedures generate large amounts of aerosols during their execution due to the instrumentation used during the procedure. Common examples of this include dental or medical handpieces, ultrasonic or sonic scalers and other high-speed instruments that use air or water to cool or flush the operative area.

These aerosols can contain infective particles of bacteria, viruses and prions. The infective particles contained in the aerosols can spread into the atmosphere surrounding the operative area causing unwanted infections in both the surgeons and their assistants. Furthermore, the aerosol can contaminate the operative room and equipment posing an infective danger to future patients and operators who utilize the room or equipment. This danger has been exacerbated by the global COVID-19 pandemic, putting medical practitioners at even greater risk.

Most clinical settings where such operations are performed have a High Volume Evacuator (HVE) system which is a rubber tube connected to a device that generates a negative pressure. Removing these aerosols via the HVE system allows for proper disposal of them and decreases the risk of transmission of various pathogens (bacteria, virus, prions) to the person performing the procedure and subsequent patients, however at present a clinical assistant is needed on hand to hold the opening of the HVE tube at the appropriate position.

It is within this context that the present invention is provided.

### SUMMARY

The present disclosure provides a manifold that can be easily attached to a medical or dental chair in which a patient sits and a flexible arm and funnel connected to the manifold and configured to receive a High Volume Evacuator (HVE) tube. The flexible arm which holds the funnel is configured to be sufficiently rigid to maintain its shape and position, allowing a clinical practitioner to leave the funnel in the appropriate place to capture aerosols emitted from a patient during a procedure use the negative pressure from the HVE. Thus, also provided herein is a method of using the disclosed manifold to remove aerosols emitted during a clinical procedure.

Thus, according to one aspect of the present disclosure there is provided a manifold for attachment to a dental or medical chair, the manifold comprising: a manifold housing having a body, a first conduit running through the body and having first and second openings, and means of attachment for securing the body to a dental or medical chair, wherein the first opening is configured to receive a High Volume Evacuator, HVE, tube.

The manifold further comprises a flexible arm having a first end and a second end connected by a second conduit, the first end of the flexible arm being connected to the second end of the first conduit, the flexible arm being configured to hold whatever shape and position it is bent into; and a funnel attachment connected to the second end of the flexible arm.

In some embodiments, the means of attachment for securing the body to a dental or medical chair is a permanent magnet housed in a flat base of the body. The magnet may be a neodymium magnet.

In some embodiments, the first opening has a threaded portion and a cap for covering the threaded portion when the manifold is not in use.

In some embodiments, one or more additional flexible arms are connected to the first conduit by a branching Y-style connector.

In some embodiments, the flexible arm is formed of a hollow length of ball-and-socket jointed tubing with the second conduit running through the hollow portion.

In some embodiments, one or more of the openings of the first and second conduits and connections therebetween are fitted with rubber gaskets to maintain an airtight seal.

In further embodiments, the funnel attachment is detachably connected to the second end of the flexible arm by a connector.

In yet further embodiments, the funnel attachment has spiral-shaped rifling on an inner surface configured to create a negative pressure vortex when connected to an HVE tube.

In yet further embodiments, the funnel attachment has a lip with a grooved gutter and a small bleed tube connecting the second conduit to an opening adjacent the lip to collect condensate collected by the grooved gutter.

Finally, in some embodiments, the funnel attachment is hyperbolic in shape.

According to another aspect of the present disclosure, there is provided a method of use of the manifold of any one of the above described embodiments to remove aerosols emitted from a patient during a medical or dental procedure.

### BRIEF DESCRIPTION OF THE DRAWINGS

Various embodiments of the invention are disclosed in the following detailed description and accompanying drawings.

FIG. 1 illustrates a first perspective view of an example configuration of the manifold of the present disclosure attached to a dental chair with the funnel attachment proximal to a patient's mouth.

FIG. 2 illustrates a second perspective view of the example configuration of the manifold of the present disclosure viewed from the rear.

FIG. 3 illustrates a third close up view of the example configuration of the manifold of the present disclosure viewed from the rear.

FIG. 4 illustrates a fourth close up view of the example configuration of the manifold of the present disclosure viewed from the side.

FIG. 5 illustrates an exploded view of the components of an example configuration of a manifold housing comprising a magnetic base for attaching to a medical or dental chair.

FIG. 6 illustrates an assembled side on view of the example configuration of the manifold housing.

FIG. 7 illustrates a perspective view of a connector for mounting a funnel attachment to the flexible arm of the manifold.

FIG. 8 illustrates a cross-sectional view of the connector of FIG. 7.

FIG. 9 illustrates a front view of an example of a specialised funnel attachment configuration suitable for use with the manifold of the present disclosure.

FIG. 10 illustrates a rear perspective view of the example specialised funnel attachment.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present disclosure relates to a manifold attachment 100 that can be attached to a medical or dental chair 200 and connected to a High Volume Evacuator (HVE) system 400 to apply a localized force of negative pressure in close proximity to a dental or medical procedure likely to generate an aerosol from a patient 300, thereby decreasing the risk of spreading aerosols containing dangerous particles to the operators, equipment and space in which a dental or medical procedure is performed. The manifold captures the infectious aerosols and directs them through the HVE system for later safe disposal according to industry accepted protocols.

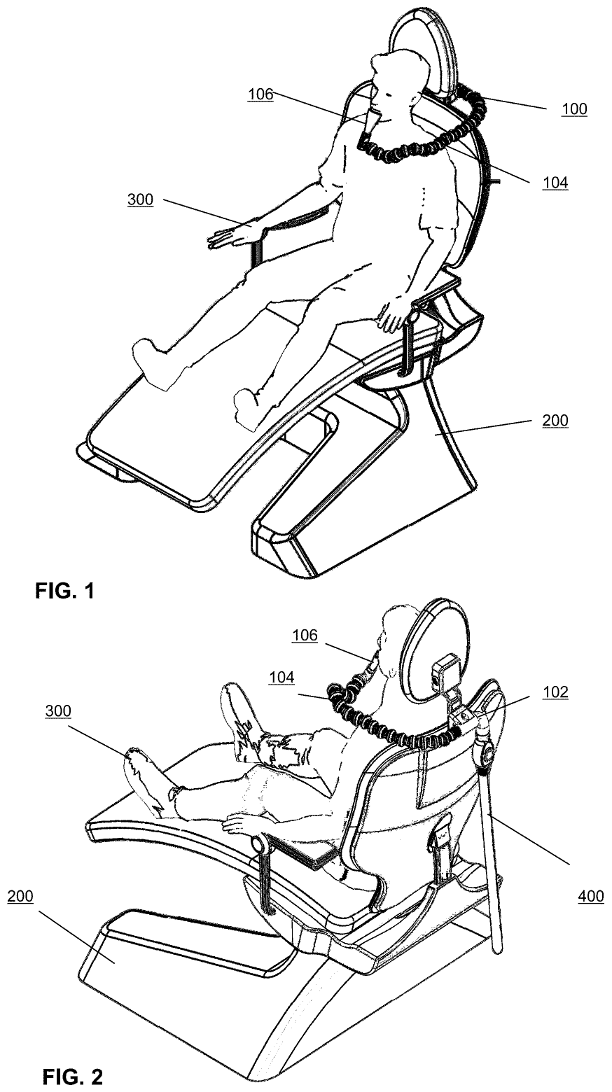

Referring to FIGS. 1 and 2, an example configuration of the manifold 100 is shown in place over the mouth of a patient 300, which includes a housing 102 which is attachable to the chair 200, a flexible arm 104, and a funnel 106 for actually capturing the aerosols.

In the present example configuration, the manifold 100 comprises a magnetised base in the manifold housing 102 and has been attached to a steel plate on the rear of the medical chair 200. Many medical and dental chairs and operatories have numerous metal plates and surfaces on which such a housing 102 could be conveniently attached. Furthermore, attaching the manifold 100 to the actual operating chair 200 advantageously means that it moves in unison with the chair if it is turned, raised, or lowered.

Most medical and dental chairs have at least one such steel plate, however for those that do not the manifold can also be provided with a suction-based attachment means as will be described below.

The flexible arm 104 which also comprises a conduit connected directly to the HVE system 400 is shown reaching around to the front of the patient, placing the funnel attachment 106 near their mouth where aerosols are most likely to be emitted.

The flexible arm is self-supporting, meaning that it is malleable but maintains whatever shape it is manipulated into and does not hang or droop, removing the need for an assistant to hold the attachment in place during a procedure. It can easily be positioned and repositioned to match the position of the patient. In some examples the arm 104 may be formed of a loc-line like tube of connected ball-and-socket joints with a hollow portion through which the conduit runs.

The funnel attachment 106 may be detachable from the flexible arm 106, allowing multiple different attachments to be used which are tailored to the type of procedure being performed. A specialised funnel attachment for maximising the amount of aerosol and condensate captured is also disclosed herein and is described below.

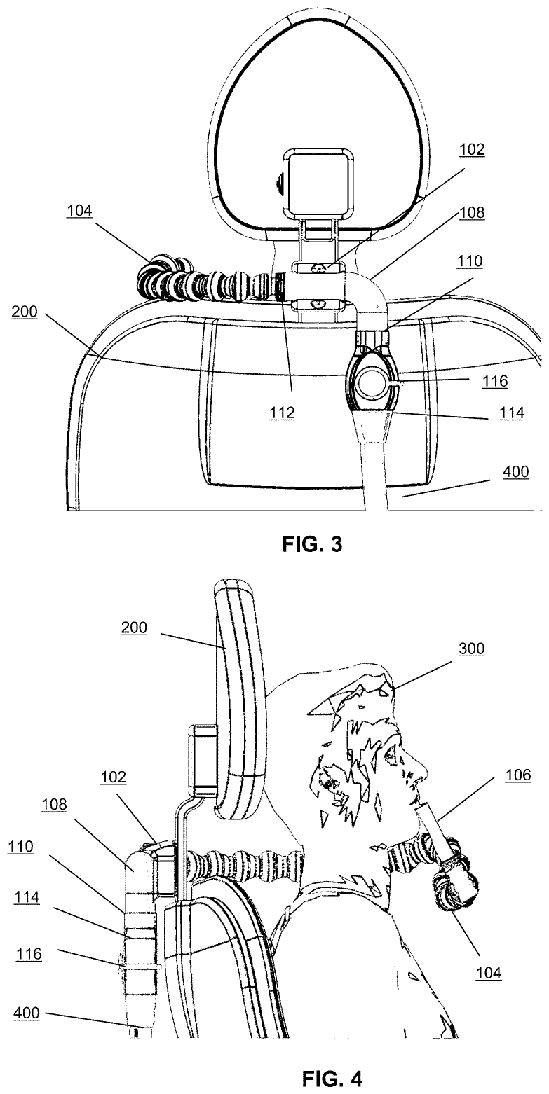

Referring to FIGS. 3 and 4, close-up views of the manifold 100 are shown from the rear and side to better illustrate the positioning of the housing 102 and funnel 106. In the illustrated configuration, the housing 102 has been attached to a metal plate on the neck of the operating chair 200.

As the tube of the HVE system 400 which provides the negative pressure is likely to be trailing vertically down to the floor, it may be beneficial for the conduit 108 that runs through the housing 102 and connects to the HVE tube 400 to be curved, with the opening 110 that connects to the HVE tube 400 thus also being vertical and reducing the moment of force applied by the weight of the dangling HVE tube.

The second opening 112 of the housing conduit 108 that interfaces with the conduit that runs through the flexible arm 104 would thus be oriented in a horizontal position, which is likely to be an effective orientation for the flexible arm 104 to reach around to the patient's mouth.

In the illustrated configuration a rubber gasket 114 connects the HVE tube to the opening 110 of the housing 102, keeping the manifold airtight to prevent the negative pressure applied from being reduced by leaks. The connection may also comprise a valve 116 for regulating or adjusting the amount of negative pressure applied according to the procedure being carried out.

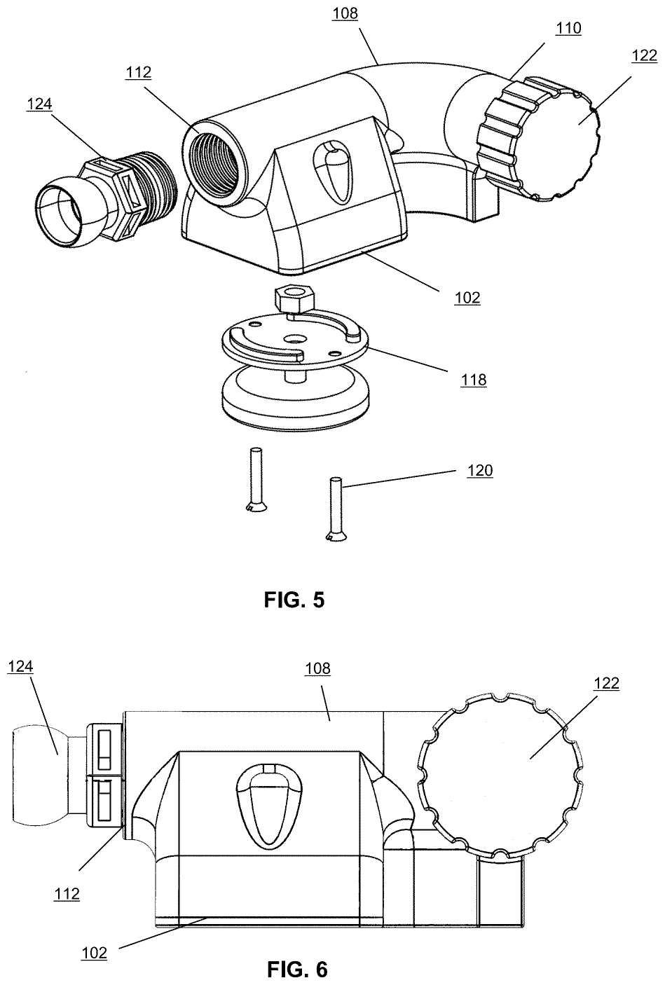

Referring to FIG. 5, an exploded view of an example configuration of the manifold housing 102 is shown in isolation.

The illustrated configuration comprises a magnetised base as described above, with a neodymium magnet disc 118 being securely fitted to a flat base of the housing by screw elements 120.

The housing conduit 108 is also shown in the curved configuration described above, with a threaded cap element 122 screwed over the first opening 110 to prevent contamination while not in use and a connector element 124 included over the second opening 112 for interfacing with the flexible arm 104.

FIG. 6 shows the same configuration of the manifold housing 102 in an assembled state.

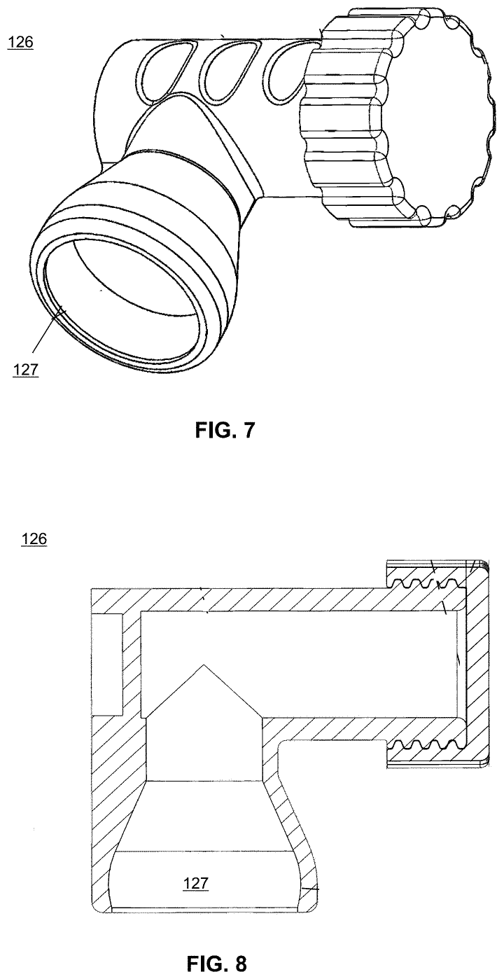

Referring to FIGS. 7 and 8, an example configuration of a connector element 126 is shown which comprises an inlet 127 for receiving the second end of the flexible arm and an opening for detachably receiving any number of different types of funnel attachments, with the funnel attachment that is connected being easily changeable to suit the type of operation being carried out.

While many different shapes and sizes of funnel can be used in combination with the disclosed manifold, in some cases it may be useful to have a funnel attachment specifically designed to maximise the amount of negative pressure applied to a local area and the amount of aerosols and condensate captured during a clinical procedure.

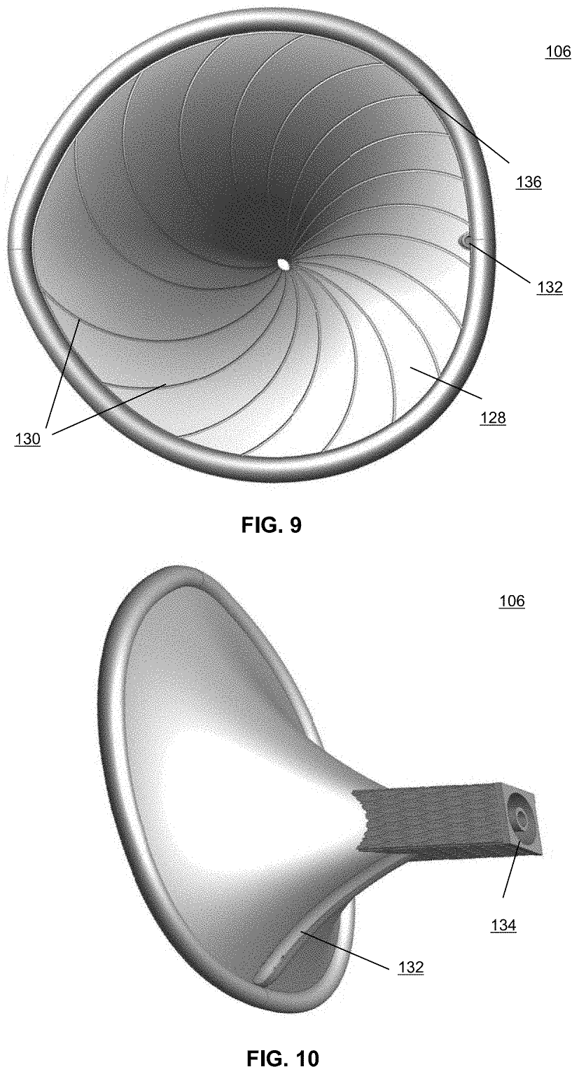

Referring to FIGS. 9 and 10, a specialised funnel attachment design suitable for use with the disclosed manifold is illustrated which achieves this object by having a wide hyperbolic shaped funnel interior surface 128, spiral-shaped rifling grooves 130 on the surface to funnel captured air into a vortex, and a draining spout 132 for capturing condensate that collects on the inner surface 128.

The funnel attachment 106 is in a hyperbolic shape with a large patient-facing inner surface 128 that tapers towards the connection to the conduit of the flexible arm 104, which it interfaces with through a suction attachment 134.

In the present example the suction attachment 134 has a rectangular outer covering with surface knurling which facilitates easy attachment of a clamp of flexible arm 104. As mentioned above, the flexible arm 104 allows for fine-tuning the position of the manifold for maximum effectiveness and moving out of the operative field for removal once the procedure has been completed.

The funnel 106 has a unique rounded lip which makes it safe in cases of contact with persons or equipment, and also features a Coanda lip profile which enhances the negative pressure. A concave groove 136 runs the circumference of the inside of the funnel lip, this serves as a condensate and liquid catchment which will conduct the fluid down the groove to the draining spout 132. Any condensed liquid or errant spray that is collected on the surface of the funnel is conveyed along the groove 136 towards draining spout 132, which is itself a small conduit connected to the main HVE negative pressure channel.

The internal surface 128 may be rifled in a spiral pattern to allow for the development of a rotating vortex of air in the funnel and in its close proximity allowing for an increase in the effectiveness of the manifold's aerosol gathering capacity.

The manifold may be made of materials that are disposable or sterilizable for safe reuse. In some examples it is manufactured from clear plastics material thereby decreasing the risk of a patient feeling claustrophobic.

The manifold may have a straight walled funnel shape. The manifold may have a cup or saucer shape. The square outer housing where the flexible arm clamp attaches may feature alternative attachments to secure the manifold to the flexible arm such as a magnetic attachment or a mechanical interlocking attachment.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the manifold attachment and method of use thereof to remove aerosols in a clinical environment have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1: A manifold for attachment to a dental or medical chair, the manifold comprising:
a manifold housing having a body, a first conduit running through the body and having first and second openings, and means of attachment for securing the body to a dental or medical chair, wherein the first opening is configured to receive a High Volume Evacuator, HVE, tube;
a flexible arm having a first end and a second end connected by a second conduit, the first end of the flexible arm being connected to the second end of the first conduit, the flexible arm being configured to hold whatever shape and position it is bent into; and
a funnel attachment connected to the second end of the flexible arm.

2: A manifold according to claim 1, wherein the means of attachment for securing the body to a dental or medical chair is a permanent magnet housed in a flat base of the body.

3: A manifold according to claim 2, wherein the magnet is a neodymium magnet.

4: A manifold according to claim 1, wherein the first opening has a threaded portion and a cap for covering the threaded portion when the manifold is not in use.

5: A manifold according to claim 1, wherein one or more additional flexible arms are connected to the first conduit by a branching Y-style connector.

6: A manifold according to claim 1, wherein the flexible arm is formed of a hollow length of ball-and-socket jointed tubing with the second conduit running through the hollow portion.

7: A manifold according to claim 1, wherein one or more of the openings of the first and second conduits and connections therebetween are fitted with rubber gaskets to maintain an airtight seal.

8: A manifold according to claim 1, wherein the funnel attachment is detachably connected to the second end of the flexible arm by a connector.

9: A manifold according to claim 1, wherein the funnel attachment has spiral-shaped rifling on an inner surface configured to create a negative pressure vortex when connected to an HVE tube.

10: A manifold according to claim 1, wherein the funnel attachment has a lip with a grooved gutter and a small bleed tube connecting the second conduit to an opening adjacent the lip to collect condensate collected by the grooved gutter.

11: A manifold according to claim 1, wherein the funnel attachment is hyperbolic in shape.

12: (canceled)
